(19) United States
(12) Patent Application Publication
(10) Pub. No.: US 2026/0207913 A1
(43) Pub. Date: Jul. 23, 2026

(54) PROTECTIVE COVER FOR IMPLANTED MEDICAL PORTS WITH IMPACT DISTRIBUTION AND VENTILATION SYSTEM

(71) Applicant: Emily Mulvaney, Denver, CO (US)

(72) Inventor: Emily Mulvaney, Denver, CO (US)

(21) Appl. No.: 19/426,267

(22) Filed: Dec. 19, 2025

Related U.S. Application Data
(60) Provisional application No. 63/747,334, filed on Jan. 20, 2025.

(51) Int. Cl. A61M 39/02

(52) U.S. Cl. CPC A61M 39/0247; A61M 2039/0258; A61M 2039/0273; A61M 2039/0288

(57) ABSTRACT

A protective cover for implanted medical ports comprises a flexible adhesive base layer in circular, ovular, hexagonal, or diamond configurations and a protective structure that redistributes impact forces away from the port. The structure rises 8-10 mm to maintain clearance above typical 5 mm port protrusions. Ventilation features including holes, slits, or apertures prevent moisture accumulation while maintaining breathability. The cover utilizes medical-grade materials with engineered deformation properties that absorb impacts up to 15 pounds-force. The disposable device features intuitive band-aid style application with removable backing tabs, enabling patients to safely engage in daily activities while maintaining port protection.

### CROSS REFERENCE TO RELATED APPLICATIONS

This application claims the benefit of priority under 35 U.S.C. § 119(e) to U.S. Provisional Ser. No. 63/747,334 , filed Jan. 20, 2025, entitled “PROTECTIVE COVER FOR IMPLANTED MEDICAL PORTS WITH IMPACT DISTRIBUTION AND VENTILATION SYSTEM,” the entire disclosure of which is hereby incorporated by reference.

### BACKGROUND OF THE INVENTION

Patients requiring long-term vascular access often receive surgically implanted ports, also known as Port-A-Caths, which are placed under the skin and connected to blood vessels to facilitate repeated access for medical treatments. While these ports provide essential medical benefits, they create significant challenges and risks for patients during daily activities outside of controlled medical settings.

A critical problem exists regarding the vulnerability of implanted ports, such as for example “Port-A-Cath” ports,” to external impacts and pressure during routine activities. The port creates a raised area under the skin, typically extending approximately 5 millimeters above the normal skin surface, with a diameter roughly equivalent to that of a quarter. This protrusion leaves the port susceptible to contact from everyday items such as clothing, seatbelts, and shower water, as well as incidental or accidental impacts during activities associated with daily living.

The risks associated with external forces on implanted ports are particularly pronounced in pediatric patients, who may engage in physical activities like sports or playground games. Impact to the port area can cause severe discomfort, potentially dislodge the port, and significantly increase infection risks. The lack of adequate protection during non-medical activities, especially activities that take place outside of a hospital, represents a substantial gap in patient care and safety.

Additionally, adult patients with implanted ports face similar challenges during routine daily activities and physical exercise. Activities such as exercising at the gym, participating in recreational sports, or even routine tasks like driving with a seatbelt can create risks of impact or pressure on the port site. These impacts can lead to the same serious complications seen in pediatric patients, including port displacement, increased infection risk, and significant discomfort that may interfere with work and daily responsibilities. The vulnerability of the port's raised profile, typically extending 5 millimeters above the skin surface, makes it susceptible to contact from everyday items such as clothing and seatbelts, highlighting the critical need for protective solutions that accommodate adult lifestyle demands.

Current solutions are primarily focused on short-term protection during medical procedures or medication administration, rather than addressing the ongoing need for protection during daily activities to allow for a less restricted life. Existing products, such as those incorporating lidocaine for numbing, are designed for brief periods of use during medical procedures and are not suitable for continuous wear or impact protection.

The medical community has long recognized the need for a solution that provides consistent protection for ports during everyday activities while maintaining patient comfort and allowing proper ventilation to prevent moisture-related complications. This need is especially critical for pediatric patients, who may have difficulty complying with protective measures and are at higher risk for accidental impacts to the port area.

### SUMMARY OF THE INVENTION

An embodiment of the present invention provides a protective cover for implanted medical ports that combines a flexible adhesive base with a protective dome structure to shield ports from external impacts while enabling comfortable daily wear. The protective cover addresses the challenges faced by patients with implanted ports during routine activities outside of medical settings.

In a preferred embodiment, the protective cover comprises an adhesive base layer made of medical-grade flexible material in a substantially circular, ovular, hexagonal, or football-diamond shaped configuration that removably attaches to the patient's skin proximal to and optionally surrounding the implanted port. The adhesive base supports a gradually sloped dome structure positioned above the port protrusion, engineered from medical-grade silicone or FDA-approved plastic with specific deformation properties that allow controlled compression under impact while preventing harmful force transmission to the underlying port.

The dome structure features a graduated slope design that creates a deformation zone, distributing impact forces over a broader area before they reach the implanted port. The material composition and thickness of the dome are specifically engineered to provide sufficient rigidity to resist ordinary contact pressures during daily activities while allowing controlled deformation under higher impact forces. This controlled deformation mechanism acts as a mechanical buffer, absorbing and redirecting impact energy away from the sensitive port site.

The adhesive base layer incorporates medical-grade adhesive materials that maintain secure attachment during normal patient activities including sleeping, showering, and exercise, while allowing for pain-free removal without skin irritation or residue. The adhesive properties are balanced to provide sufficient adherent strength to keep the protective cover in place during typical wear periods of several days to a week, while remaining gentle enough for patients with sensitive skin or compromised tissue integrity around the port site.

The protective cover further incorporates a ventilation system comprising strategically positioned apertures or breathable material sections that allow air circulation and moisture vapor transmission while maintaining the protective barrier function of the dome structure. This ventilation architecture prevents the accumulation of perspiration and moisture beneath the cover, reducing the risk of skin maceration, bacterial growth, and associated complications during extended wear periods. The shape and dimensions of various embodiments are optimized to provide sufficient adhesive surface area while accommodating different port locations and body contours.

An example embodiment includes an impact distribution system integrated into the dome structure, featuring radially oriented reinforcement elements that actively redirect applied forces or unanticipated impacts away from the sensitive port area. The system maintains effectiveness for impacts up to 15 pounds-force while preventing direct force transmission to the port area.

A preferred embodiment incorporates a ventilation system comprising ventilation features that may include one or more of: holes, perforations, slits, apertures, or openings. These ventilation features may be strategically positioned on the dome structure, at lateral sides of the dome structure, or at the transition zone between the dome structure and the adhesive base layer. The ventilation features are distributed to provide air exchange while maintaining structural integrity of the protective structure. These ventilation features work in conjunction with the moisture vapor transmission properties of the adhesive base layer to maintain appropriate humidity levels and prevent moisture accumulation between the device and skin surface. The ventilation features may comprise various configurations including: an array of circular holes distributed across the dome surface, elongated slits positioned at lateral regions of the protective structure, apertures at the periphery where the dome meets the adhesive base, or combinations thereof. The size, shape, and positioning of these ventilation features are engineered to balance air circulation requirements with the structural demands of impact protection.

The protective cover invention incorporates several technical innovations that collectively address the shortcomings of prior art solutions. It utilizes an intuitive band-aid style application method with removable backing tabs for sequential placement, enabling easy application and removal by patients or caregivers without specialized training. The dome's graduated slope design, combined with engineered material properties, creates a deformation zone that progressively absorbs impact energy before it reaches the port site. The ventilation architecture prevents moisture accumulation and associated skin complications while maintaining the protective barrier. These integrated features create a comprehensive solution that addresses both immediate impact protection and long-term wearability concerns.

### BRIEF DESCRIPTION OF THE FIGURES

FIG. 1a depicts a side view of the protective cover in an embodiment.

FIG. 1b depicts a top down view of the protective cover in an embodiment.

FIG. 2a depicts a perspective view of the protective cover in an embodiment.

FIG. 2b depicts a perspective view of separate protective covers of different shapes in embodiments.

FIG. 3 depicts a perspective view of an alternative embodiment of the protective cover.

FIG. 4a depicts an intended use of a protective cover being placed upon a wearer's skin over a port in an embodiment.

FIG. 4b depicts an exemplary placement of a protective cover over a port in an embodiment.

FIG. 5 depicts a cross-sectional cutout view of the protective cover showing the protective structure and flexible adhesive base layer in an embodiment.

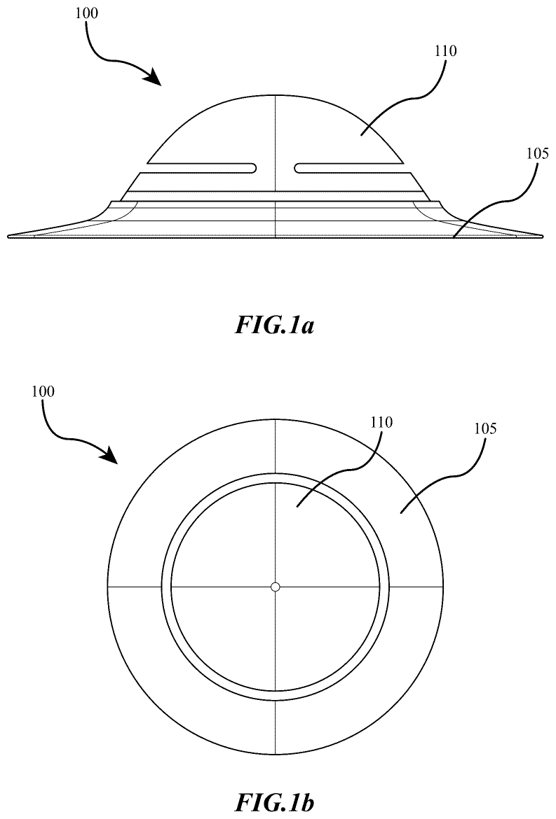

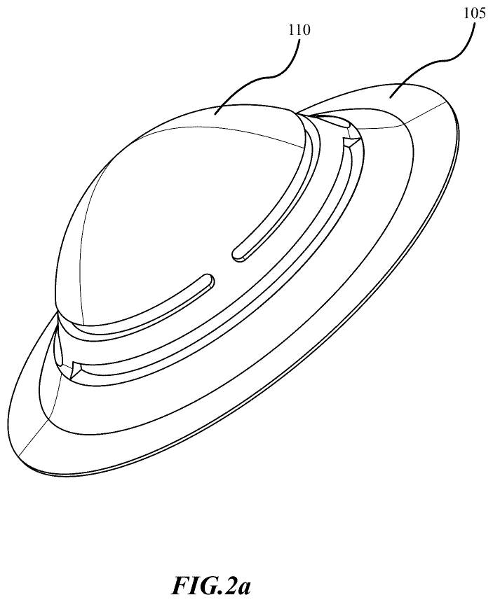

### DETAILED DESCRIPTION OF THE INVENTION

An embodiment of the present invention provides a protective cover 100 for implanted medical ports that addresses the challenges faced by patients during daily activities. The protective cover 100 comprises a flexible adhesive portion 105 and a protective structure 110 configured to shield the implanted port 300 from external impacts while allowing for comfortable everyday wear.

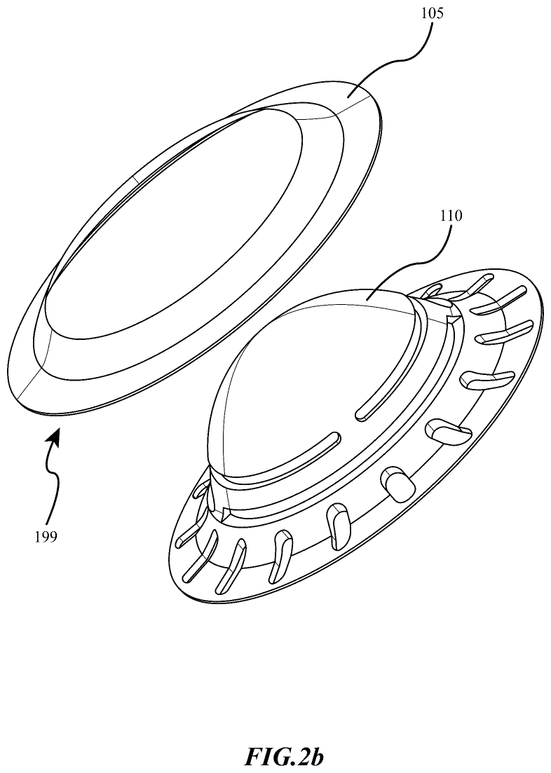

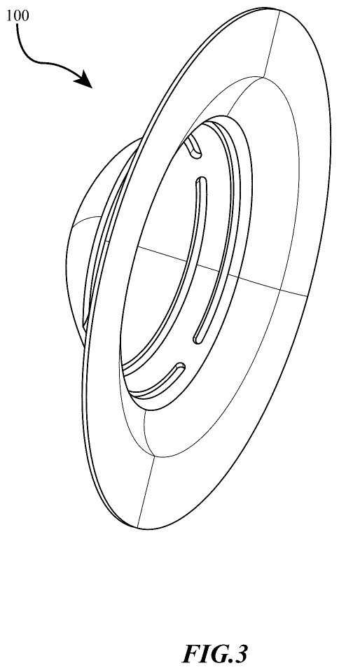

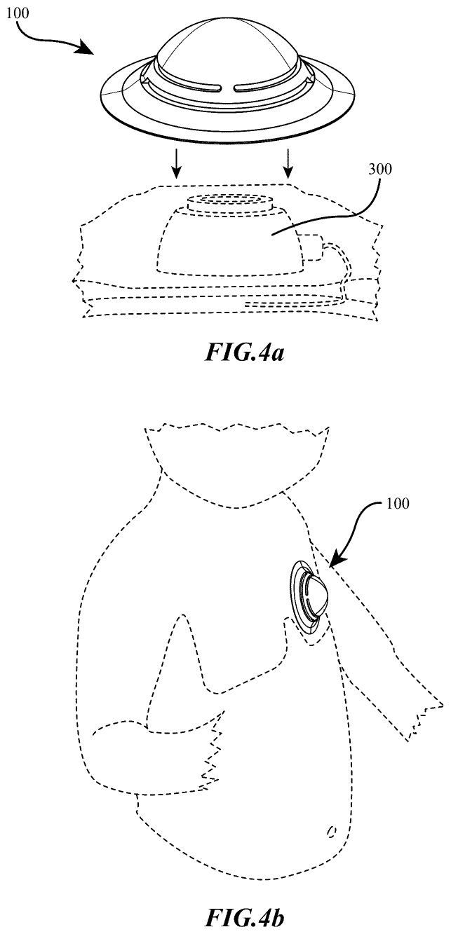

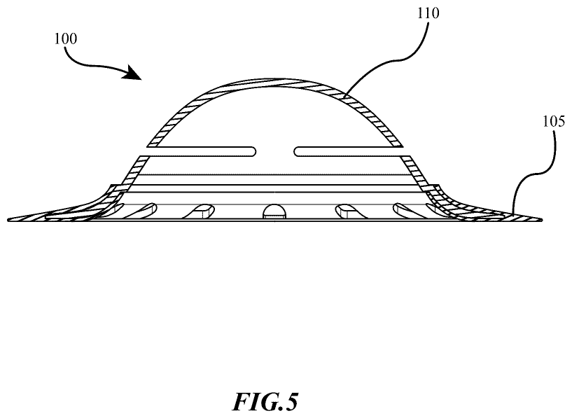

In a preferred embodiment of the invention, the protective cover 100 includes a flexible adhesive portion 105 comprising a shaped adhesive base layer, optionally shaped in a substantially circular, hexagonal or ovular form, that removably attaches to the patient's skin surrounding the implanted port. In various embodiments, the alternatively shaped protective cover 199 such as that depicted in FIG. 2b can resemble any number of shapes, including shapes designed to correspond to irregular port 300 sizes, placement, or patient anatomy.

In accordance with various embodiments, the flexible adhesive portion 105 supports a protective structure 110, comprising a gradually sloped dome structure positioned above the port 300 protrusion on a patient's skin during intended use. The dome structure comprises a biocompatible, medical-grade silicone elastomer or FDA-approved thermoplastic material with a Shore A hardness rating between 40-60, providing optimal balance between rigidity for protection and flexibility for comfort. Alternative embodiments may utilize medical-grade polypropylene or polyethylene materials that meet ISO 10993 biocompatibility standards for skin-contacting devices.

The protective structure 110, in various embodiments comprising a domed geometrical structurer featuring a gradually sloped profile rising from the peripheral edges at approximately 15-20 degrees to a central height of 8-10mm above the skin surface, providing adequate clearance above the typical 5 mm port protrusion. This gradual slope enables effective force distribution while maintaining a low profile for patient comfort. The transition between the dome and the adhesive base in an embodiment incorporates a radius of approximately 3-4mm to prevent stress concentration.

The dome structure includes a circular central protective region with a diameter of approximately 35 mm (1.38 inches), which exceeds the typical quarter-sized port diameter of 24.26 mm (0.955 inches), providing complete coverage of standard port dimensions with adequate margin. The total dome structure measures approximately 45 mm in diameter at its base to ensure proper force distribution and edge sealing.

The dome structure in accordance with various embodiments utilizes a graduated wall thickness profile, varying from approximately 2.0 mm at the apex to 1.5 mm at the periphery. This graduated thickness profile, combined with the dome's geometric properties, creates a controlled deformation pattern that maintains structural integrity while dispersing impact energy.

Alternative size embodiments can be provided to accommodate varying port dimensions, as port sizes may differ between patients of different ages and sizes.

The dome structure is engineered with specific deformation characteristics that prevent contact with the underlying port even under direct impact, maintaining a minimum 2 mm clearance above the port protrusion when under maximum deflection.

While the protective structure 110 provides impact protection in a preferred embodiment, alternative geometric configurations may be utilized to achieve similar protective benefits. The protective structure 110 in alternative embodiments may comprise various three-dimensional shapes including pyramidal, conical, or multi-faceted geometric forms that maintain adequate clearance above the port while providing impact distribution capabilities. These alternative geometries can incorporate the same graduated wall thickness profile varying from approximately 2.0 mm at the apex to 1.5 mm at the periphery, combined with radially oriented reinforcement features that guide impact forces outward along predetermined paths.

Regardless of the specific geometric configuration chosen, the protective structure 110 maintains the core functional requirements of deflecting under loads up to 15 pounds-force while maintaining a minimum 2 mm clearance above the port protrusion. The structure rises to a central height of 8-10mm above the skin surface to accommodate the typical 5 mm port protrusion, with the specific slope and contour varying based on the chosen geometric form. Each alternative configuration incorporates the ventilation features and impact distribution system, with the structural elements engineered to channel force vectors radially outward at angles between 15-30 degrees relative to the skin surface.

The protective structure 110, whether dome-shaped or utilizing alternative geometry, maintains a circular central protective region measuring approximately 35 mm in diameter to ensure complete coverage of standard port dimensions, with the total structure measuring approximately 45 mm in diameter at its base for proper force distribution and edge sealing.

In one embodiment, the invention incorporates ventilation features comprising an array of precisely positioned micro-perforations distributed across the protective structure 110. These ventilation holes measure approximately 0.5-1.0 mm in diameter and are arranged in a radial pattern with spacing of 3-5 mm between adjacent holes, providing optimal air circulation while maintaining structural integrity of the protective dome.

In this embodiment, the ventilation system includes strategically positioned zones, with a higher concentration of perforations in areas prone to moisture accumulation. The central region includes 8-12 primary ventilation holes arranged in a circular pattern approximately 15 mm from the dome's center, while secondary ventilation zones near the peripheral edges contain 12-16 additional holes. This configuration creates a natural convection effect that promotes moisture evacuation and prevents sweat pooling beneath the device.

In an example of the invention, the breathability features work in conjunction with the moisture vapor transmission properties of the flexible adhesive portion 105. The combined ventilation system maintains a relative humidity level below 80% in the space between the device and skin surface during normal use. The ventilation design allows for air exchange rates of approximately 0.5-1.0 cubic centimeters per minute under typical wearing conditions, sufficient to prevent moisture accumulation while maintaining the protective function of the device. The size and placement of the ventilation features are engineered to prevent any compromise of the dome's structural integrity or impact resistance capabilities.

In one embodiment, the ventilation features comprise an array of precisely positioned micro-perforations distributed across the protective structure 110. These ventilation holes measure approximately 0.5-1.0 mm in diameter and are arranged in a radial pattern with spacing of 3-5 mm between adjacent holes, providing optimal air circulation while maintaining structural integrity of the protective dome.

In an alternative embodiment, the ventilation system comprises elongated slits positioned at lateral sides of the protective structure 110, extending radially outward from the central protective region. These slits measure approximately 3-8 mm in length and 0.5-1.5 mm in width, providing air circulation while maintaining structural integrity of the protective structure 110. The slit configuration provides an alternative ventilation approach that may be preferred for certain manufacturing processes or patient preferences while achieving functionally equivalent air exchange performance.

The side slits may be positioned at the transition zone between the dome structure and the adhesive base layer, approximately 15-20 mm from the dome's center. This configuration allows for moisture evacuation through lateral air flow while preserving the dome's apex structural integrity for impact distribution. In an exemplary embodiment, 4-8 slits are arranged in a radial pattern around the protective structure 110, with each slit oriented to facilitate convective air flow from the central region outward to the periphery. The lateral positioning of the slits maintains the structural strength of the dome's apex while providing equivalent or superior ventilation performance compared to hole-based configurations. The slits may be formed through laser cutting, die cutting, or molding processes during manufacturing.

The protective cover 100 in accordance with an embodiment is intended as a disposable device with an expected use duration of 24-72 hours before replacement. The single-use design ensures optimal adhesive performance and maintains hygienic conditions throughout the wear period. The device in accordance with an exemplary embodiment is packaged individually in sealed, sterile pouches. In an embodiment, the inventor has observed that such configuration of the device exhibits a shelf life of approximately 24 months when stored at room temperature.

In accordance with various embodiments, the dimensions and structural properties of the protective cover 100 are engineered to accommodate standard port dimensions that typically extend approximately 5 mm above the skin surface with a diameter equivalent to a quarter (24.26mm/0.955 inches).

The dome structure in accordance with an embodiment features specific deformation characteristics that prevent contact with the underlying port even under impact conditions. The materials of the protective structure 110 and its geometry in an example are chosen to deflect under a load of up to 15 pounds-force while maintaining a minimum 2 mm clearance above the port protrusion. The protective structure's 110 structural properties enable it to return to its original shape after deformation within normal use parameters.

The protective structure 110 in an embodiment incorporates a circular central protective region measuring approximately 35 mm (1.38 inches) in diameter, which provides complete coverage exceeding the standard quarter-sized port diameter with adequate margin. The total protective structure 110 measures approximately 45 mm in diameter at its base in an embodiment to ensure proper force distribution.

The protective structure 110 in accordance with various examples utilizes a graduated wall thickness profile, varying from approximately 2.0 mm at the apex to 1.5 mm at the periphery, combined with radially oriented reinforcement features that guide impact forces outward along predetermined paths. When an impact occurs, the structural features channel the force vectors radially outward at angles between 15-30 degrees relative to the skin surface, spreading the impact force over an area approximately 3-4 times larger than the initial point of contact.

Alternative size embodiments can be provided to accommodate varying port 300 dimensions, as port 300 sizes may differ between patients of different ages and sizes, while maintaining the core structural properties that prevent contact between the protective structure 110 and port 300 during impact.

In an embodiment of the invention, the flexible adhesive portion 105 comprises a substantially circular, ovular, hexagonal, or diamond shaped configuration that extends outward from a central region designed to overlay the implanted port. Various shape configurations provide optimal adhesion distribution and minimize edge lifting during wear. In embodiments utilizing elongated shapes such as football-diamond or hexagonal configurations, the elongated ends may be oriented along the primary stress lines of the skin to maintain secure attachment during patient movement. Circular and ovular embodiments provide uniform radial adhesion distribution suitable for various port locations and body contours.

A preferred embodiment of the flexible adhesive portion 105 utilizes a medical-grade, flexible adhesive material similar to Tegaderm™, comprising a thin, transparent polyurethane film coated with a hypoallergenic, pressure-sensitive acrylic adhesive. The adhesive material maintains its adherent properties while allowing moisture vapor transmission, enabling extended wear times of up to 7 days in an intended exemplary method of use while maintaining skin integrity. The flexible adhesive portion's 105 thickness in an embodiment measures approximately 1.0 mm to provide sufficient bonding strength while maintaining flexibility.

In a preferred embodiment, the flexible adhesive portion 105 comprises a substantially planar, flexible base formed from medical-grade materials approved for prolonged skin contact. The total surface area of the flexible adhesive portion 105 in various embodiments provides sufficient adherent surface to support the protective dome structure while distributing attachment forces across a broad skin area. In embodiments utilizing elongated configurations such as hexagonal or football-diamond shapes, the adhesive portion may measure approximately 4 inches along its major axis and 2.5 inches along its minor axis. In circular or ovular embodiments, the adhesive portion may have a diameter of approximately 3-4 inches, providing comparable adhesive surface area. The flexible nature of the material allows the adhesive portion 105 to conform to the body's contours, maintaining secure attachment during patient movement while remaining comfortable during extended wear periods.

In a preferred embodiment of the invention, the protective structure 110 comprises a biocompatible, medical-grade silicone elastomer or FDA-approved thermoplastic material with a Shore A hardness rating between 40-60, providing optimal balance between rigidity for protection and flexibility for comfort. Alternative embodiments may utilize medical-grade polypropylene or polyethylene materials that meet ISO 10993 biocompatibility standards for skin-contacting devices.

The protective structure 110 of an example embodiment features a gradually sloped profile rising from the peripheral edges at approximately 15-20 degrees to a central height of 8-10 mm, providing adequate clearance above the typical 5 mm port protrusion. This gradual slope enables effective force distribution while maintaining a low profile for patient comfort. The transition between the dome and the adhesive base incorporates a radius of approximately 3-4mm to prevent stress concentration.

In an embodiment of the invention, the protective structure 110 incorporates specific deformation characteristics engineered to maintain protection during impact from an object. The dome structure in an example comprises a biocompatible, medical-grade silicone elastomer or FDA-approved thermoplastic material with a Shore A hardness rating between 40-60, providing an optimal balance of rigidity for protection while maintaining flexibility for comfort. Alternative materials include medical-grade polypropylene or polyethylene that meet ISO 10993 biocompatibility standards for skin-contacting devices.

As described herein, in accordance with an example wherein the protective structure 110 comprises a dome geometry, the dome's structural design incorporates a graduated wall thickness profile varying from approximately 2.0 mm at the apex to 1.5 mm at the periphery. This graduated thickness, combined with the dome's geometric properties, creates a controlled deformation pattern that maintains structural integrity while effectively dispersing impact energy.

The force redistribution mechanism in accordance with various embodiments operates through an integrated structural design featuring radially oriented reinforcement elements within the dome material. When an impact occurs at any point on the dome surface, these structural features channel the force vectors radially outward at angles between 15-30 degrees relative to the skin surface. This redistribution pattern spreads the impact force over an area approximately 3-4 times larger than the initial point of contact, significantly reducing peak force transmission to the underlying port.

The dome structure in accordance with an embodiment maintains a minimum 2 mm clearance above the port protrusion even under maximum deflection from impacts up to 15 pounds-force. The material properties enable the dome to return to its original shape after deformation within these normal use parameters, ensuring consistent protection during daily activities.

A preferred embodiment includes a circular central protective region with a diameter of approximately 35 mm (1.38 inches), exceeding the typical quarter-sized port diameter of 24.26 mm (0.955 inches), providing complete coverage of standard port dimensions with adequate margin. The total dome structure measures approximately 45 mm in diameter at its base to ensure proper force distribution and edge sealing. The dome's interior height of 8-10mm provides sufficient clearance for the 5 mm port protrusion while maintaining necessary air space for ventilation and impact absorption.

In various embodiments of the invention, the impact distribution system comprises an integrated structural design that actively redirects applied forces away from the sensitive port area. The structural body incorporates a series of radially oriented reinforcement features within the dome material, configured to guide impact forces outward along predetermined paths toward the peripheral edges of the device.

In an example, the graduated thickness profile of the dome combined with the dome's geometric properties, creates a controlled deformation pattern that maintains structural integrity while dispersing impact energy. The dome structure's rigidity is engineered to resist localized deformation while allowing controlled flexing across its entire surface.

In an example of the invention, the force redistribution mechanism operates through a combination of material properties and geometric design. When an impact occurs at any point on the dome surface, the structural features channel the force vectors radially outward at angles between 15-30 degrees relative to the skin surface. This redistribution pattern spreads the impact force over an area approximately 3-4 times larger than the initial point of contact, significantly reducing peak force transmission to the underlying port 300. The system maintains effectiveness for impacts up to 15 pounds-force while preventing direct force transmission to the area immediately proximal to the port 300.

In an embodiment of the invention, the interface elements incorporate a disposable design with an expected use duration of 24-72 hours before replacement. The protective cover 100 is manufactured as a single-use device with materials and construction methods optimized for cost-effective production while maintaining medical-grade quality standards. The disposable nature ensures optimal adhesive performance and maintains hygienic conditions throughout the wear period.

A preferred embodiment utilizes a band-aid style application method featuring an intuitive peel-and-stick approach. In an exemplary embodiment, the application system includes tabs on the removable backing, allowing for placement similar to common medical dressings. The backing material may be divided into sections that can be removed individually, enabling precise positioning over the port site while maintaining sterility of the adhesive surface. The application process requires no specialized training or medical assistance, allowing patients or caregivers to easily apply and remove the device.

In an example of the invention, the protective cover 100 is designed for off-the-shelf availability through standard medical supply channels and retail pharmacies. The device is packaged individually in sealed, sterile pouches with a shelf life of approximately 24 months when stored at room temperature. Each package in an exemplary embodiment contains complete application instructions and is labeled with relevant sizing information, lot numbers, and expiration dates in compliance with FDA labeling requirements. The packaging configuration allows for bulk distribution in boxes of 10-30 units, suitable for both institutional and individual patient use.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

I claim:

1. A protective cover for an implanted medical port comprising:
a flexible adhesive base layer configured for removable attachment to a patient's skin;
a protective structure coupled to the adhesive base layer and positioned to extend over an implanted port, wherein the protective structure comprises a gradually sloped profile configured to redistribute impact forces away from the implanted port; and
ventilation features configured to allow air exchange, wherein the ventilation features comprise at least one of: holes, perforations, slits, or apertures positioned on or within the protective structure.

2. The protective cover of claim 1, wherein the ventilation features comprise holes distributed across the protective structure.

3. The protective cover of claim 1, wherein the ventilation features comprise slits positioned at lateral sides of the protective structure.

4. The protective cover of claim 1, wherein the ventilation features comprise both holes on the protective structure and slits at lateral regions of the protective structure.

5. The protective cover of claim 2, wherein the holes comprise:
primary ventilation holes arranged in a circular pattern approximately 15mm from a center of the protective structure; and
secondary ventilation holes positioned near peripheral edges of the protective structure.

6. The protective cover of claim 2, wherein the holes measure approximately 0.5-1.0 mm in diameter and are arranged in a radial pattern with spacing of 3-5 mm between adjacent holes.

7. The protective cover of claim 3, wherein the slits measure approximately 3-8 mm in length and 0.5-1.5 mm in width.

8. The protective cover of claim 3, wherein the slits are positioned at a transition zone between the protective structure and the adhesive base layer, approximately 15-20 mm from a center of the protective structure.

9. The protective cover of claim 1, wherein the flexible adhesive base layer comprises:
a shaped configuration selected from circular, ovular, hexagonal, and football-diamond forms; and
a medical-grade adhesive material configured to maintain adherent properties while allowing moisture vapor transmission.

10. The protective cover of claim 1, wherein the protective structure comprises:
a biocompatible material selected from medical-grade silicone elastomer and FDA-approved thermoplastic;
a central height between 8-10 mm; and
a circular central protective region having a diameter of approximately 35 mm.

11. The protective cover of claim 1, wherein the protective structure is configured to:
deflect under a load of up to 15 pounds-force; and
maintain a minimum clearance of 2 mm above the implanted port when under maximum deflection.

12. The protective cover of claim 1, wherein the protective structure comprises:
a graduated wall thickness varying from approximately 2.0 mm at an apex to 1.5 mm at a periphery; and
radially oriented reinforcement features configured to guide impact forces outward.

13. The protective cover of claim 12, wherein the radially oriented reinforcement features are configured to channel force vectors radially outward at angles between 15-30 degrees relative to a skin surface.

14. The protective cover of claim 1, wherein the ventilation features work in conjunction with moisture vapor transmission properties of the flexible adhesive base layer to maintain a relative humidity level below 80% between the protective cover and skin surface.

15. The protective cover of claim 1, further comprising:
a peel-away backing divided into multiple sections; and
non-adherent tabs configured to facilitate removal of the protective cover.

16. The protective cover of claim 1, wherein:
the protective structure is configured for single use with an expected wear duration of 24-72 hours; and
the protective cover is packaged in sealed, sterile pouches having a shelf life of approximately 24 months at room temperature.

17. A method of protecting an implanted medical port comprising:
providing a protective cover having a flexible adhesive base layer and a protective structure with ventilation features;
removing a sectioned backing from the adhesive base layer; and
applying the protective cover over an implanted port such that the protective structure extends over and maintains clearance above the implanted port.

18. The method of claim 17, wherein the protective structure comprises:
a gradually sloped profile rising at approximately 15-20 degrees from peripheral edges;
a central height sufficient to maintain clearance above a 5 mm port protrusion; and
ventilation features configured to prevent moisture accumulation.

19. The method of claim 17, wherein the ventilation features comprise at least one of: holes distributed across the protective structure, slits positioned at lateral sides of the protective structure, or a combination thereof.

20. The method of claim 17, further comprising:
wearing the protective cover for a duration of 24-72 hours during daily activities; and
removing and replacing the protective cover after the wear duration.
